(19) United States
(12) Patent Application Publication
(10) Pub. No.: US 2024/0180794 A1
(43) Pub. Date: Jun. 6, 2024

(54) STICK-TYPE OIL-DISPERSION COSMETIC COMPOSITION COMPRISING POWDER

(71) Applicant: COSMAX, INC., Gyeonggi-do (KR)

(72) Inventors: Yoon Nam CHO, Gyeonggi-do (KR); Hyeon Joo LIM, Gyeonggi-do (KR); Jin Goo JI, Gyeonggi-do (KR); Myeong Sam PARK, Seoul (KR)

(21) Appl. No.: 17/918,967

(22) PCT Filed: Nov. 5, 2021
(86) PCT No.: PCT/KR2021/016003

§ 371 Date: Oct. 14, 2022

(30) Foreign Application Priority Data
Sep. 16, 2021 (KR) 10-2021-0123846

(51) Int. Cl. A61K 8/02; A61K 8/04; A61Q 19/00

(52) U.S. Cl. CPC A61K 8/0229; A61K 8/04; A61Q 19/00; A61K 2800/10

(57) ABSTRACT

The present invention relates to a stick-type oil-dispersion cosmetic composition containing a powder, a wax, and an oil, wherein a stick-type oil-dispersion cosmetic composition, which mitigates powder failing of existing powder and has reduced oiliness, is prepared by mixing the powder and the wax at an optimum ratio and adding the oil thereto, thereby implementing a stick-type oil-dispersion cosmetic composition containing a powder and showing excellent effects in moisturizing and a soft feeling of use.

### TECHNICAL FIELD

This application claims priority to Korean Patent Application No. 10-2021-0123846 filed in the Korean Intellectual Property Office on 16 Sep. 2021, the disclosure of which is incorporated herein by reference.

The present disclosure relates to a stick-type oil-dispersion cosmetic composition containing a powder, a wax, and an oil.

### BACKGROUND ART

Powder can eliminate the sticking feeling of cosmetic products through its distinctive feeling of use, and powder with oil absorbing ability can serve to protect the skin exposed to sweat and moisture, such as a part between folded legs or a crotch part.

Conventional powder uses a powder formulation of cosmetic composition containing a powder such as silica or starch, but the use of such a formulation causes an inhalation problem due to powder falling and has inconvenience in that lotion, cream, or the like needs to be additionally applied to improve moisturizing.

In general, cosmetic products need to be applied using the palm or fingers, and thus cause problems that the products are consumed in unnecessary sites or leave a feeling of irritation, so that a stick-type cosmetic composition is presented as an alternative to solve such problems. Such a stick-type cosmetic composition is convenient to use and easy to carry. However, typical stick-type cosmetic compositions are anhydrous and are composed of hydrophobic wax, oil, and the like, and thus are effective in preventing moisture loss, but have a fault of having a lot of oiliness.

There is an urgent need to develop a stick-type oil-dispersion cosmetic composition containing a powder which mitigates the inconvenience of powder failing of existing powder, eliminates oiliness of a stick-type cosmetic composition, and has excellent moisturizing and soft feeling.

### DISCLOSURE OF INVENTION

### Technical Problem

The present inventors have made intensive research efforts to produce a stick-type oil-dispersion cosmetic composition, which mitigates powder failing of existing powder and has reduced oiliness.

Accordingly, an aspect of the present disclosure is provided to a stick-type oil-dispersion cosmetic composition containing a powder, a wax, and an oil.

### Solution to Problem

Hereinafter, the present disclosure will be described in more detail.

In accordance with an aspect of the present disclosure, there is provided a stick-type oil-dispersion cosmetic composition containing a powder, a wax, and an oil.

In the present disclosure, the weight ratio of the powder and the wax may be 1:1 to 8:1, 1:1 to 7:1, 1:1 to 6:1, or 1:1 to 5:1, and for example, 4:1, but is not limited thereto.

In the present disclosure, the powder may be at least one selected from the group consisting of aluminum starch octenylsuccinate, polymethylsilsesquioxane, silica, and Zea mays (corn) starch, but is not limited thereto.

In the present disclosure, the powder may be contained, relative to the total cosmetic composition, in a content of 0.1 to 50.0 wt %, 0.1 to 45.0 wt %, 0.1 to 40.0 wt %, 0.1 to 35.0 wt %, 0.1 to 30.0 wt %, 5.0 to 50.0 wt %, 5.0 to 45.0 wt %, 5.0 to 40.0 wt %, 5.0 to 35.0 wt %, 5.0 to 30.0 wt %, 10.0 to 50.0 wt %, 10.0 to 45.0 wt %, 10.0 to 40.0 wt %, 10.0 to 35.0 wt %, 10.0 to 30.0 wt %, 15.0 to 50.0 wt %, 15.0 to 45.0 wt %, 15.0 to 40.0 wt %, 15.0 to 35.0 wt %, 15.0 to 30.0 wt %, 20.0 to 50.0 wt %, 20.0 to 45.0 wt %, 20.0 to 40.0 wt %, 20.0 to 35.0 wt %, 20.0 to 30.0 wt %, 25.0 to 50.0 wt %, 25.0 to 45.0 wt %, 25.0 to 40.0 wt %, 25.0 to 35.0 wt %, or 25.0 to 30.0 wt %, and for example, 30.0 wt %, but is not limited thereto.

In the present disclosure, the weight ratio of aluminum starch octenylsuccinate and silica may be 1:1 to 5:1, 1:1 to 4:1, 1:1 to 3:1, or 1:1 to 2:1, and for example, 1:1, but is not limited thereto.

In the present disclosure, the wax may be at least one selected from the group consisting of Helianthus annuus (sunflower) seed wax, candelilla wax, microcrystalline wax, bees wax, Copernicia cerifera (carnauba) wax, cerecin, synthetic wax, synthetic beeswax, and ozokerite, but is not limited thereto.

In the present disclosure, the wax may be contained, relative to the total cosmetic composition, in a content of 0.1 to 20.0 wt %, 0.1 to 17.0 wt %, 0.1 to 15.0 wt %, 0.1 to 13.0 wt %, 0.1 to 10.0 wt %, 0.1 to 8.0 wt %, 3.0 to 20.0 wt %, 3.0 to 17.0 wt %, 3.0 to 15.0 wt %, 3.0 to 13.0 wt %, 3.0 to 10.0 wt %, 3.0 to 8.0 wt %, 6.0 to 20.0 wt %, 6.0 to 17.0 wt %, 6.0 to 15.0 wt %, 6.0 to 13.0 wt %, 6.0 to 10.0 wt %, or 6.0 to 8.0 wt %, and for example, 7.5 wt %, but is not limited thereto.

In the present disclosure, the oil may be at least one selected from the group consisting of Helianthus annuus (sunflower) seed oil, Pyrus malus seed oil, camellia oil, Prunus amygdalus dulcis (sweet almond) oil, Jojoba esters, triethylhexanoin, polyglyceryl-2 triisostearate, and octyldodecanol, but is not limited thereto.

In the present disclosure, the oil may be contained, relative to the total cosmetic composition, in a content of 10.0 to 80.0 wt %, 10.0 to 75.0 wt %, 10.0 to 70.0 wt %, 10.0 to 65.0 wt %, 10.0 to 60.0 wt %, 20.0 to 80.0 wt %, 20.0 to 75.0 wt %, 20.0 to 70.0 wt %, 20.0 to 65.0 wt %, 20.0 to 60.0 wt %, 30.0 to 80.0 wt %, 30.0 to 70.0 wt %, 30.0 to 65.0 wt %, 30.0 to 60.0 wt %, 40.0 to 80.0 wt %, 40.0 to 75.0 wt %, 40.0 to 70.0 wt %, 40.0 to 65.0 wt %, 40.0 to 60.0 wt %, 50.0 to 80.0 wt %, 50.0 to 75.0 wt %, 50.0 to 70.0 wt %, 50.0 to 65.0 wt %, 50.0 to 60.0 wt %, 55.0 to 80.0 wt %, 55.0 to 75.0 wt %, 55.0 to 70.0 wt %, or 55.0 to 65.0 wt %, and for example, 60.0 wt %, but is not limited thereto.

### Advantageous Effects of Invention

The present disclosure is directed to a stick-type oil-dispersion cosmetic composition containing a powder, a wax, and an oil, wherein a stick-type oil-dispersion cosmetic composition, which mitigates powder failing of existing powder and has reduced oiliness, is prepared by mixing the powder and the wax at an optimum ratio and adding the oil thereto, thereby implementing a stick-type oil-dispersion cosmetic composition containing a powder and showing excellent effects in moisturizing and a soft feeling of use.

### BEST MODE FOR CARRYING OUT THE INVENTION

The present disclosure is directed to a stick-type oil-dispersion cosmetic composition containing a powder, a wax, and an oil.

### MODE FOR CARRYING OUT THE INVENTION

Hereinafter, the present disclosure will be described in more detail by the following examples. However, these exemplary embodiments are used only for illustration, and the scope of the present disclosure is not limited by these exemplary embodiments.

### Preparation Example 1: Preparation of Examples

Examples 1 and 2 were prepared by varying the type and content of each ingredient as shown in Table 1 below.

TABLE 1

| NO | Use | Ingredient | Example 1 | Example 2 |
|---|---|---|---|---|
| 1 | Powder | Aluminum starch | 15.0 | 25.0 |
| | | octenylsuccinate | | |
| 2 | | Silica | 15.0 | 25.0 |
| 3 | Wax | Helianthus annuus | 7.5 | 7.5 |
| | | (sunflower) seed wax | | |
| 4 | Oil | camellia oil | 20.0 | 20.0 |
| 5 | Others | Sunflower seed oil | 42.5 | 22.5 |

| | | |
|---|---|---|
| Total (unit: wt %) | 100.0 | 100.0 |

Specifically, all raw materials were weighed in beakers according to each ingredient and content thereof shown in Table 1, and then uniformly mixed at 25 rpm for 5 minutes by Agi-Mixer while warmed at 75-85° C. Finally, air bubbles were removed, and the mixtures were poured into containers and cooled to prepare stick-type oil-dispersion cosmetic compositions of Examples 1 and 2.

### Preparation Example 2: Preparation of Comparative Examples

Comparative Examples 1 to 4 were prepared by varying the type and content of each ingredient as shown in Table 2 below.

TABLE 2

| | | | Comparative | Comparative | Comparative | Comparative |
|---|---|---|---|---|---|---|
| NO | Use | Component | Example 1 | Example 2 | Example 3 | Example 4 |
| 1 | Powder | Polymethylsilsesquioxane | — | 30 | — | — |
| 2 | | Aluminum starch | — | — | 30.0 | — |
| | | octenylsuccinate | | | | |
| 3 | | Silica | — | — | — | 30.0 |
| 4 | Wax | Helianthus annuus | 7.5 | 7.5 | 7.5 | 7.5 |
| | | (sunflower) seed wax | | | | |
| 5 | Oil | camellia oil | 20.0 | 20.0 | 20.0 | 20.0 |
| 6 | | Helianthus annuus | 72.5 | 42.5 | 42.5 | 42.5 |
| | | (sunflower) seed oil | | | | |

| | | | | |
|---|---|---|---|---|
| Total (unit: wt %) | 100.0 | 100.0 | 100.0 | 100.0 |

Specifically, all raw materials were weighed in beakers according to each ingredient and content thereof shown in Table 2, and then uniformly mixed at 25 rpm for 5 minutes by an Agi-Mixer while warmed at 75-85° C. Finally, air bubbles were removed, and the mixtures were poured into containers and cooled to prepare stick-type oil-dispersion cosmetic compositions of Comparative Examples 1 to 4.

### Test Example 1 Evaluation of Formulation Stability

Examples 1 and 2 and Comparative Examples 1 and 4 were investigated for formulation stability. Each oil-dispersion cosmetic composition was evaluated for formulation stability under conditions of 4° C., room temperature, 37° C., 45° C., and cycle (the temperature was changed every 24 hours three times with a storage temperature of −4° C.) for 1 day, 5 days, 1 week, 2 weeks, and 4 weeks, and the results are shown in Table 3 below. The evaluation criteria were as follows.

### <Evaluation Criteria>

⊚; Stable
Δ; Slight unstable
X; unstable

TABLE 3

| | | Example | Example | Comparative | Comparative | Comparative | Comparative |
|---|---|---|---|---|---|---|---|
| | | 1 | 2 | Example 1 | Example 2 | Example 3 | Example 4 |
| 1 Day | 4° C. | ⊚ | ⊚ | ⊚ | ⊚ | ⊚ | ⊚ |
| | Room | ⊚ | ⊚ | ⊚ | ⊚ | ⊚ | ⊚ |
| | temperature | | | | | | |
| | 37° C. | ⊚ | ⊚ | ⊚ | ⊚ | ⊚ | ⊚ |
| | 45° C. | ⊚ | ⊚ | ⊚ | ⊚ | ⊚ | ⊚ |
| | Cycle | ⊚ | ⊚ | ⊚ | ⊚ | ⊚ | ⊚ |
| 5 Days | 4° C. | ⊚ | ⊚ | ⊚ | ⊚ | ⊚ | ⊚ |
| | Room | ⊚ | ⊚ | ⊚ | ⊚ | ⊚ | ⊚ |
| | temperature | | | | | | |
| | 37° C. | ⊚ | ⊚ | ⊚ | ⊚ | ⊚ | ⊚ |
| | 45° C. | ⊚ | ⊚ | Δ | ⊚ | ⊚ | ⊚ |
| | Cycle | ⊚ | ⊚ | Δ | ⊚ | ⊚ | ⊚ |
| 1 Week | 4° C. | ⊚ | ⊚ | ⊚ | ⊚ | ⊚ | ⊚ |
| | Room | ⊚ | ⊚ | ⊚ | ⊚ | ⊚ | ⊚ |
| | temperature | | | | | | |
| | 37° C. | ⊚ | ⊚ | Δ | ⊚ | ⊚ | ⊚ |
| | 45° C. | ⊚ | ⊚ | Δ | ⊚ | ⊚ | ⊚ |
| | Cycle | ⊚ | ⊚ | X | ⊚ | ⊚ | ⊚ |
| 2 Weeks | 4° C. | ⊚ | ⊚ | ⊚ | ⊚ | ⊚ | ⊚ |
| | Room | ⊚ | ⊚ | ⊚ | ⊚ | ⊚ | ⊚ |
| | temperature | | | | | | |
| | 37° C. | ⊚ | ⊚ | Δ | ⊚ | ⊚ | ⊚ |
| | 45° C. | ⊚ | ⊚ | X | ⊚ | ⊚ | ⊚ |
| | Cycle | ⊚ | ⊚ | X | ⊚ | ⊚ | ⊚ |
| 4 Weeks | 4° C. | ⊚ | ⊚ | ⊚ | ⊚ | ⊚ | ⊚ |
| | Room | ⊚ | ⊚ | ⊚ | ⊚ | ⊚ | ⊚ |
| | temperature | | | | | | |
| | 37° C. | ⊚ | ⊚ | Δ | ⊚ | ⊚ | ⊚ |
| | 45° C. | ⊚ | ⊚ | X | ⊚ | ⊚ | ⊚ |
| | Cycle | ⊚ | ⊚ | X | ⊚ | ⊚ | ⊚ |

As can confirmed in Table 3, Examples 1 and 2 and Comparative Examples 2 to 4 showed a favorable stable formulation even 4 weeks after preparation. However, Comparative Example 1 was observed to show a formulation unstable from 5 days after preparation.

It can be therefore confirmed that Comparative Example 1 not containing a powder showed an unstable formulation, and a powder needs to be contained for a stable formation.

### Test Example 2 Sensory Evaluation

Thirty professional female subjects aged 25 to 45 were divided into two groups, and Examples 1 to 2 and Comparative Examples 1 to 4 were appropriately adjusted for each group and applied onto the face and the hand, and then evaluated for spreadability and powderiness according to the following evaluation criteria.

In the evaluation criteria, the spreadability was evaluated on whether the prepared stick-type oil-dispersion cosmetic composition was smoothly spread on the face and the hand. The powderiness was evaluated on whether there was a sticky feeling or soft feeling on the hand when the prepared stick-type oil-dispersion cosmetic composition was applied to the face and the hand and then allowed to absorb thereinto.

The evaluation of each item was carried out according to a 5-point scale including quite excellent (5 points), excellent (4 points), fair (3 points), poor (2 points), and very poor (1 point). As for spreadability, a higher score indicates better spreadability, and as for the powderiness, a higher score indicates a less sticky and softer feeling. The evaluation results are shown in Table 4.

TABLE 4

| | Example | Example | Comparative | Comparative | Comparative | Comparative |
|---|---|---|---|---|---|---|
| Classification | 1 | 2 | Example 1 | Example 2 | Example 3 | Example 4 |
| Spreadabilty | 4.2 | 3.5 | 4.0 | 2.0 | 4.0 | 3.0 |
| Powderiness | 4.4 | 3.6 | 0.0 | 2.9 | 3.2 | 4.0 |

As can be confirmed in Table 4, Example 1 was evaluated to be excellent in both spreadability and powderiness compared with Comparative Examples 1 to 4. Comparative Example 1 was evaluated to have excellent spreadability but very low powderiness, and Comparative Example 2 was evaluated to be low in both spreadability and powderiness. Comparative Example 3 was evaluated to have excellent spreadability but somewhat low powderiness, and Comparative Example 4 was evaluated to have low spreadability but high powderiness.

It can be therefore confirmed that, when applied to the skin, the combined use of silica and aluminum starch octenylsuccinate at a ratio of 1:1 showed excellent spreadability and powdery feeling compared with the use of silica and aluminum starch octenylsuccinate alone.

### INDUSTRIAL APPLICABILITY

The present disclosure relates to a stick-type oil-dispersion cosmetic composition containing a powder, a wax, and an oil.

What is claimed is:

1. A stick-type oil-dispersion cosmetic composition comprising a powder, a wax, and an oil.

2. The stick-type oil-dispersion cosmetic composition of claim 1, wherein the weight ratio of the powder and the wax is 1:1 to 8:1.

3. The stick-type oil-dispersion cosmetic composition of claim 1, wherein the powder is at least one selected from the group consisting of aluminum starch octenylsuccinate, polymethylsilsesquioxane, silica, and Zea mays (corn) starch.

4. The stick-type oil-dispersion cosmetic composition of claim 1, wherein the powder is contained in a content of 0.1 to 50.0 wt % relative to the total cosmetic composition.

5. The stick-type oil-dispersion cosmetic composition of claim 3, wherein the weight ratio of aluminum starch octenylsuccinate and silica is 1:1 to 5:1.

6. The stick-type oil-dispersion cosmetic composition of claim 1, wherein the wax is at least one selected from the group consisting of Helianthus annuus (sunflower) seed wax, candelilla wax, microcrystalline wax, bees wax, Copernicia cerifera (carnauba) wax, cerecin, synthetic wax, synthetic beeswax, and ozokerite.

7. The stick-type oil-dispersion cosmetic composition of claim 1, wherein the wax is contained in a content of 0.1 to 20.0 wt % relative to the total cosmetic composition.

8. The stick-type oil-dispersion cosmetic composition of claim 1, wherein the oil is at least one selected from the group consisting of Helianthus annuus (sunflower) seed oil, Pyrus malus seed oil, camellia oil, Prunus amygdalus dulcis (sweet almond) oil, Jojoba esters, triethylhexanoin, polyglyceryl-2 triisostearate, and octyldodecanol.

9. The stick-type oil-dispersion cosmetic composition of claim 1, wherein the oil is contained in a content of 10.0 to 80.0 wt % relative to the total cosmetic composition.
